(19) United States
(12) Patent Application Publication
(10) Pub. No.: US 2023/0086586 A1
(43) Pub. Date: Mar. 23, 2023

(54) Use of a combination of an amino acid and elemental calcium as neurotransmitters precursors

(71) Applicant: Eric Marchewitz, Lake Orion, MI (US)

(72) Inventor: Eric Marchewitz, Lake Orion, MI (US)

(21) Appl. No.: 17/956,676

(22) Filed: Sep. 29, 2022

Related U.S. Application Data
(60) Provisional application No. 63/244,525, filed on Sep. 15, 2021.

(51) Int. Cl. C12P 13/00; A61K 33/06; A61K 33/30; A61K 31/198

(52) U.S. Cl. CPC C12P 13/005; A61K 33/06; A61K 33/30; A61K 31/198

(57) ABSTRACT

This invention relates a method of combining an amino acid, with one of an elemental calcium, potassium, sodium, zinc and magnesium ion as an oral supplement for the purpose of passing the amino acid through the blood brain barrier for the delivery of specific amino acids with a higher PH, for enhancing athletic performance, for reducing fat, and/or as an anti-aging agent.

### FIELD OF THE INVENTION

This invention relates a method of combining an amino acid, with a salt including one of a calcium, potassium, sodium, zinc and magnesium ion as an oral supplement for the purpose of passing the amino acid through the blood brain barrier for the delivery of specific amino acids with a higher PH, for enhancing athletic performance, increasing neurotransmitters, for reducing fat, and/or as an anti-aging agent.

### BACKGROUND OF THE INVENTION

Healthy brain function is dependent on an ample supply of neurotransmitters. These neurotransmitters are produced by the body, and additionally, diet also plays a role in neurotransmitter production. Amino acids act as a neurotransmitter or neurotransmitter precursor. However, current amino acids have a low PH causing the amino acid to have poor bioavailability in the brain and are typically taken in their free form. The blood brain barrier is semi-resistant to free form amino acids and nutrients, and thus limits the amount of amino acids passing through to the brain tissue to be used as precursors to neurotransmitters. By bonding an amino acid with a mineral one of an elemental calcium, potassium, sodium, zinc and magnesium ion, the acidity of the amino acid is lowered, increasing passage of the amount of amino acids into the brain. These combinations specifically are intended to increase the amount of these compounds that can pass the blood brain barrier by increasing the PH of the amino group. There exists a need for the delivery of specific amino acids in combination with minerals such as an elemental calcium potassium, sodium, zinc and/or magnesium ion with a higher PH, for enhancing athletic performance, for reducing fat, and/or as an anti-aging agent.

Disclosed in this invention are methods for combining an amino acid, with a mineral such as one of an elemental calcium, potassium, sodium, zinc or magnesium ion, as an oral supplement for the purpose of passing through the blood brain barrier for the delivery of specific amino acids with a higher PH to act as neurotransmitter precursors.

### SUMMARY OF THE INVENTION

In accordance with a first aspect, methods are disclosed for combining an amino acid, with a mineral to create a salt such as one of a calcium, potassium, sodium, zinc or magnesium ion. as an oral supplement. for the purpose of increased passage through the blood brain barrier for the delivery of specific amino acids with a higher PH.

From the foregoing disclosure and the detailed description set forth below of various preferred embodiments of the invention it will be apparent to those skilled in the art that the disclosed invention provides a significant advance in the methods of delivering amino acids for supplying increased neurotransmitters or neurotransmitter precursors to the brain. The unique formulas disclosed herein will provide a substantial advantage over traditional oral administration of amino acids. Additional features and advantages of various preferred embodiments will be better understood in view of the detailed description provided below.

### DETAILED DESCRIPTION

It will be apparent to those skilled in the art, that is, to those who have a knowledge or experience in this area of technology that many variations are possible for the method of orally administering a combination of representative amino acids or N-acetylated amino acids which may act as a neurotransmitter such as:

Either the N-Acetyl or Free Form Amino Acid of one of the following:

### L-Tryptophan

### L-5-Hydroxy-Tryptophan

### L-Tyrosine

### L-Phenylalanine

### D-Phenylalanine

Gamma-aminobutyric acid
L-Glutamic acid

### L-Dopa

### L-Glutamic Acid

### L-Aspartate

In the preferred embodiment the amino acid would be Gamma-aminobutyric acid:

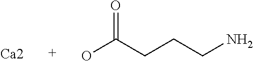

All of the compound formulations disclosed herein would be administered orally. Alternatively, the formulations might be administered via IV, or sublingually. In the preferred embodiment a selected amino acid would be ionic bonded with an elemental calcium ion in a liquid or solid carrier. In an alternative embodiment the selected amino acid would be bonded with one of a potassium, sodium, zinc and/or magnesium ion. In the preferred embodiment these various compounds would preferably be administered orally mixed with a liquid carrier in appropriate unit doses.

The preferred amount of each of the active amino acid and one of an elemental calcium, potassium, sodium, zinc and magnesium ion ingredients within the formulation that is to be orally administered would depend on various factors such as the specific drug or dietary supplement molecular weight that needs to be utilized to obtain the desired result. The concentration of the amino acid compound in the formulation might be between 80% and 90%. It might be between 70% and 80%. The preferred concentration would be 75%. The concentration of the one of an elemental calcium, potassium, sodium, zinc and magnesium ion in the formulation might be between 19% and 20%. It might be between 17% and 22%. The preferred concentration would be 19.5%.

### Example Formulation One

250 mg total weight of elemental magnesium ionic bonded with Gamma-aminobutyric acid in a gelatin capsule for oral administration.

### Example Formulation Two

100 mg total weight of elemental calcium bonded ionically with Gamma-aminobutyric acid in a gelatin capsule for oral administration.

### Example Formulation Three

500 mg total weight elemental magnesium bonded ionically with L-Tryptophan

From the foregoing disclosure and detailed description of certain preferred embodiments, it will be apparent that various modifications, additions, and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of administering orally a combined ionic bonded Amino Acid with one or more of an elemental calcium, potassium, sodium, zinc and magnesium ion for the purpose of increasing penetration of the amount of an amino acid through the blood brain barrier to increase neurotransmitter production.

2. The method of claim 1 wherein the Amino Acid consists of Gamma-aminobutyric acid.

3. The method of claim 1 wherein the Amino Acid consists of one or more of L-Hydroxy-Tryptophan, L-Dopa, L-Tyrosine, Acetyl-L-Tyrosine, and L-Glutamic Acid.

4. The method of claim 1 wherein the administered combination can be utilized for enhancing athletic performance, for reducing fat, increasing neurotransmitters and/or as an anti-aging agent.
